(19) United States
(12) Patent Application Publication
(10) Pub. No.: US 2024/0206929 A1
(43) Pub. Date: Jun. 27, 2024

(54) RESORBABLE MATERIAL ACTIVATED MECHANISMS

(71) Applicants: Children's Hospital Medical Center, Cincinnati, OH (US); Brigham Young University, Provo, UT (US)

(72) Inventors: Bethany M. Parkinson, State College, PA (US); Brandon Sargent, Provo, UT (US); Victor Garcia, Cincinnati, OH (US); Larry L. Howell, Orem, UT (US)

(21) Appl. No.: 18/567,708

(22) PCT Filed: Jun. 7, 2022
(86) PCT No.: PCT/US2022/032521

§ 371 Date: Dec. 6, 2023

Related U.S. Application Data
(60) Provisional application No. 63/197,645, filed on Jun. 7, 2021.

(51) Int. Cl. A61B 17/80; A61B 17/00; A61B 17/56

(52) U.S. Cl. CPC A61B 17/8076; A61B 17/8004; A61B 2017/00004; A61B 2017/00477; A61B 2017/00526; A61B 2017/564

(57) ABSTRACT

A device using resorbable materials to actuate, such as a device (10) for repairing pectus excavatum in a patient through activation of a bioresorbable material (40, 52).

### CROSS-REFERENCE TO RELATED APPLICATION

The present application claims priority to U.S. Provisional Patent Application Ser. No. 63/197,645, filed Jun. 7, 2021, the disclosure of which is expressly incorporated herein by reference.

### BACKGROUND AND SUMMARY OF THE DISCLOSURE

The present application relates to mechanical actuation via resorbable materials and, more particularly, to resorbable material activated mechanisms for use in a variety of applications, including medical applications such as in repairing pectus excavatum.

The majority of known mechanisms use some input to actuate. Examples of inputs could include motor torque, electricity, input loading, or heat. Passive mechanisms, or a mechanism that actuates without a specific input, are less common. The present disclosure relates to passive mechanisms utilizing resorbable materials.

Resorbable materials are materials that dissolve when exposed to certain environmental conditions. Most common among resorbable materials are water-soluble materials. Resorbable materials have been used extensively to provide temporary mechanisms or structures that resorb into their environment rather than requiring retrieval. Resorbable materials have not been used extensively as an actuation method for mechanisms. The use of resorbable materials to activate mechanisms is the focus of this disclosure.

Principal to the present disclosure is the use of stored energy. A mechanism is placed in a configuration in which it has stored energy. The most easily applicable energy would be strain energy through the use of compliant segments deflected from their initial position. The resorbable material is then used to restrain the mechanism in the energy-stored configuration. The mechanism is then placed in its intended use environment, one where the environment acts as a solvent for the resorbable restraint. As the resorbable material dissolves, the restraining force is either gradually or instantaneously removed and stored energy is used to actuate the mechanism. Therefore no additional inputs are required to activate or actuate the mechanism once placed in the working environment. This provides a distinct advantage for those applications where inputs are difficult to provide. This is shown, in particular, in an illustrative medical implant use for pectus excavatum repair.

A bioresorbable material activated pectus bar to be used for pectus excavatum repair solves several problems of previous methods. First, it provides for a more gradual (and therefore less painful) correction than the popular instantaneous correction Nuss procedure. Second, it maintains the low invasiveness of the Nuss procedure, as compared to the high-invasiveness of other methods such as the Ravitch procedure. The ability to use different bioresorbable materials as the activation for the bar also means that the rate of the bar's deployment is widely alterable pre-operation, which means that each patient could receive a different rate of pectus excavatum repair, providing additional benefit through tailorability. The bar can correct over time with no input required by surgical staff, removing the need for additional surgeries to actuate mechanisms that could have provided a similar gradual correction.

The illustrative device of the present disclosure is not necessarily incorporated into or used in combination with a computer program, although mathematical analyses of both the bar for pectus excavatum repair and for more general resorbable material activated mechanisms have been performed to validate its effectiveness.

Resorption-driven mechanisms have the ability to actuate a mechanism without an active input. As a material resorbs into the working environment, the energy stored in the system will be released and the mechanism will self-actuate. This passive activation removes the need for user input, which is of greatest benefit when user input is difficult, dangerous, expensive, or impossible.

The ability for the actuation to be gradual or all-at-once provides additional benefit. The wide variety of resorbable materials and the research done on these materials provides design freedom for resorption times and mechanics.

Illustrative applications that provide specific benefit include medical implants to avoid the need for additional surgeries to change the configuration of mechanisms, space where actuation and deployment mechanisms are expensive or massive or have reliability issues, or water sources such as deep ocean. The ability to automatically activate could also find specific benefit in reactive systems, such as alarms or valves that open or close or trigger alarms when exposed to a given solvent. To demonstrate a particular benefit in detail, the ability of a gradually changing medical implant for the correction of chest wall deformities is demonstrated.

As noted above, the most common current methods of surgery to correct pectus excavatum (PE) are the Ravitch and Nuss procedures. Both are an immediate method of repair, which require either resection of chest wall tissue or a sustained force of upwards of 230 N applied to the sternum of the patient.

The Ravitch procedure, first performed by Dr. Ravitch in 1949, was the fundamental popularized operation for the correction of PE. It involves cutting the costal cartilage, which holds the sternum in place relative to the rib cage. However, the regenerated cartilage is often “thin, irregular, and commonly rigid”, “irregular and incomplete, occasionally producing a somewhat unstable chest,” and “limits the depth of lung expansion”, so the postoperative flexibility of the chest wall is restricted. The invasiveness of this procedure lead to the development of the now standard approach, the Nuss Procedure.

The Nuss Procedure was performed for the first time in 1987 by Dr. Nuss. It involves creating a laceration on either side of the chest, using a tunneling instrument to insert a tube through the thoracic cavity, using the tube to guide a convex metal bar through the cavity, and rotating the bar until it pushes up against the sternum and pushes it out to flatten the chest. Two or three bars may be used if the indentation in the chest wall is too large for one. The Nuss Procedure provides distinct advantages to the Ravitch method. Primarily, it requires no cartilage resection and rather focuses on helping the patient's body remodel the chest wall naturally. Incision sizes are reduced and moved to the lateral sides of the chest. The Nuss Procedure, however, is highly painful due to the instantaneous correction of the chest wall. Research has suggested that a gradual approach would correct the deformity without acute pain; this is also the approach taken to move bone in other fields, such as orthodontia.

The difficulties involved in designing a gradual corrective device include keeping the device similar enough to the original bar used in the Nuss Procedure for surgeon adoption and engineering an actuation method for the device that will not require additional surgery. The present disclosure presents a novel use of resorbable materials in a bar to be used in a variation of the Nuss Procedure that produces a gradual and customizable correction of PE.

Illustrative bioresorbable materials are nontoxic to the body, dissolve slowly in the presence of bodily fluids, and change mechanical properties (losing mechanical strength and/or volume) as they dissolve. Bioresorbable materials have been a common topic for recent research, especially because of their potential to improve medical procedures. Bioresorbable implants surpass their non-resorbable counterparts in two main ways: first, by eliminating the need for retrieval surgery (they are designed to completely dissolve over time); and second, by allowing for more dynamic treatment, where a load can be gradually transferred from the implant to the surrounding bone or tissue as part of the healing process.

While the majority of the illustrative corrective implant could still contain some non-bioresorbable materials and could therefore still require a retrieval surgery, the ability to gradually transfer a load to the surrounding bone is extremely useful here. The gradual transfer is predicted to significantly decrease the pain characteristic of the Nuss Procedure, and because the bioresorbable bar is still based on the bar used in the Nuss Procedure, it would maintain the minimal invasiveness of the Nuss Procedure.

The use of resorbable materials to actuate mechanisms could have many illustrative applications. A few proposed embodiments of this technology include:
Slow release of drugs (medical implant embodiment), timed release of nutrients or other fluids/solids into water sources through the actuation of a pre-strained syringe or like mechanism;
Flip a switch or activate some bistable release when exposed to the solvent, such as opening a drain when water reaches a given height to prevent flooding;
Expand a rod or plate, such as for use in scoliosis, bone lengthening, expanding fusion cages or orthodontics;
Lift a weight or gradually exert greater force, such as in pectus bar application; Rotary mechanism, open or close a lid or valve, or twist a rod;
Unlock mechanisms to allow for deployment, such as via the outgassing of a material in space applications; and
Alarm systems that activate when exposed to a solvent and open or close valves or other systems.

All these illustrative embodiments could be all-at-once transition as the material fails structurally, or a gradual transition from one state to the next, depending on the resorption mechanics of the material used.

According to an illustrative embodiment of the present disclosure, a resorbable material activated device includes a support having a first end section, an opposing second end section, and a center section intermediate the first end section and the second end section. A first resorbable material is positioned intermediate the first end section and the center section of the support. A second resorbable material is positioned intermediate the second end section and the center section of the support. The shape of the support changes in response to dissolving of the first and second resorbable materials.

According to another illustrative embodiment of the present disclosure, a resorbable material activated device includes a compliant segment having opposing first and second ends, and a resorbable material operably coupled to the compliant segment intermediate the first and second ends. At least one of the shape, position or configuration of the compliant segment changes in response to dissolving of the resorbable material.

According to a further illustrative embodiment of the present disclosure, a device for repairing pectus excavatum in a human body includes a support having a first end section, and opposing second end section, and a center section intermediate the first end section and second end section, the support formed of a biocompatible material. A first bioresorbable material is positioned intermediate the first end section and the center section of the support, and a second bioresorbable material is positioned intermediate the second end section and the center section of the support. The center section moves from a first position to a second position in response to dissolving of the first and second bioresorbable materials to exert a force against a sternum of the human body.

According to another illustrative embodiment of the present disclosure, a method of repairing pectus excavating in a human body includes the steps of providing a support having a first end section, an opposing second end section, and a center section intermediate the first end section and the second end section. The method further includes providing a first bioresorbable material intermediate the first end section and the center section of the support, and providing a second bioresorbable material intermediate the second end section and the center section of the support. The method further includes the steps of inserting the support within a rib cage of the human body, and dissolving the first and second bioresorbable materials resulting in the changing of the shape of the support in response to dissolving of the first and second bioresorbable materials.

According to another illustrative embodiment of the present disclosure, a resorbable material activated device includes a first compliant arm extending between a first end and a second end, the first compliant arm including a first curved portion and a second curved portion intermediate the first end and the second end, the first curved portion facing an opposite direction from the second curved portion, and a second compliant arm extending between a first end and a second end, the second compliant arm including a first curved portion and a second curved portion intermediate the first end and the second end, the first curved portion facing an opposite direction from the second curved portion. The second ends of the first compliant arm and the second compliant arm are positioned a first distance apart from each other in a deformed mode, and the second ends of the first compliant arm and the second compliant arm are positioned a second distance apart from each other in a natural, undeformed mode, the second distance being greater than the first distance. A resorbable material insert is positioned intermediate the second ends of the first compliant arm and the second compliant arm in the deformed mode. The resorbable material insert is removed from intermediate the second ends of the first compliant arm and the second compliant arm in the natural, undeformed mode.

According to another illustrative embodiment of the present disclosure, a resorbable material activated device includes an arcuate arm having a first compliant section defining a first pocket, and an end effector supported by the arcuate arm. A first resorbable material insert is supported within the first pocket in a deformed mode, and removed from the first pocket in a natural, deformed mode. The end effector is in a first position in the deformed mode, and the end effector is in a second position in the natural, undeformed mode.

Additional features and advantages of the present disclosure will become apparent to those skilled in the art upon consideration of the following detailed description of the illustrative embodiments exemplifying the disclosure as presently perceived.

### BRIEF DESCRIPTION OF THE DRAWINGS

The detailed description of the drawings particularly refers to the accompanying figures in which:

FIG. 1 is an exploded perspective view of an illustrative shape-change mechanism, in the form of a corrective bar, including a support and top plates with channels for receiving bioresorbable inserts, shown in an activated mode with an undeformed position;

FIG. 2 is a perspective view of the corrective bar of FIG. 1, showing the support and the top plates assembled without bioresorbable inserts;

FIG. 3 is a cross-sectional view taken along line 3-3 of FIG. 2, including a representative bioresorbable insert;

FIG. 4 is a perspective view of a further illustrative shape-change mechanism, in the form of a corrective bar, shown in a stored energy mode with bioresorbable inserts and in a deformed position;

FIG. 5 is a perspective view of the illustrative corrective bar of FIG. 4, shown in an activated mode with the bioresorbable inserts dissolved and in a natural, undeformed position;

FIG. 6 is a perspective view of the illustrative corrective bar of FIG. 4, shown inserted through human ribs;

FIG. 7 is an illustrative view of a corrective bar similar to that of FIG. 1, with bioresorbable inserts shown under test conditions simulating the force of a human sternum, such that the mechanism is in a natural, undeformed position;

FIG. 8 is another illustrative view of the bar of FIG. 7, showing the bioresorbable inserts partially dissolved;

FIG. 9 is another illustrative view of the bar of FIG. 7, showing the bioresorbable inserts partially dissolved;

FIG. 10 is another illustrative view of the bar of FIG. 7, showing the bioresorbable inserts completely dissolved, such that the mechanism is in an activated mode with an undeformed position;

FIG. 11 is a side elevational view of a further illustrative shape-change mechanism, shown in a deformed position;

FIG. 12 is a side elevational view of the shape-change mechanism of FIG. 11, shown in a natural, undeformed position;

FIG. 13 is a diagrammatic view of an illustrative dog-bone shaped resorbable material insert for use in the shape-change mechanism of FIG. 11;

FIG. 14 is a side elevational view of a further illustrative shape-change mechanism, shown in the deformed position;

FIG. 15 is a side elevational view of the shape-change mechanism of FIG. 14, shown in a natural, undeformed position;

FIG. 16 is a perspective view of a further illustrative shape-change mechanism configured to translate resorption of resorbable material to rotation, shown in a deformed position; and

FIG. 17 is a perspective view of the shape-change mechanism of FIG. 16, shown in a natural, undeformed position.

### DETAILED DESCRIPTION OF THE DRAWINGS

For the purposes of promoting an understanding of the principles of the present disclosure, reference will now be made to the embodiments illustrated in the drawings, which are described herein. The embodiments disclosed herein are not intended to be exhaustive or to limit the invention to the precise form disclosed. Rather, the embodiments are chosen and described so that others skilled in the art may utilize their teachings. Therefore, no limitation of the scope of the claimed invention is thereby intended. The present invention includes any alterations and further modifications of the illustrated devices and described methods and further applications of principles in the invention which would normally occur to one skilled in the art to which the invention relates.

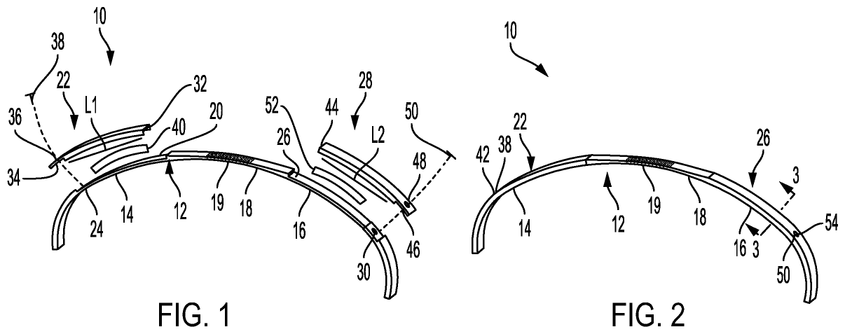

With reference initially to FIGS. 1-2, an illustrative shape-change mechanism of the present disclosure is shown in the form of a corrective device or bar 10 for repairing pectus excavatum in a human. The illustrative bar 10 includes an arcuate lower support 12 including a first end section 14, a second end section 16, and a middle or center section 18 defining a center 19. The first end section 14 illustratively includes a cutout 20 to receive a first top plate 22 and an aperture 24 to attach the support 12 and the top plate 22. The second end section 16 includes a cutout 26 to receive a second top plate 28 and an aperture 30 to attach the lower support 12 and the top plate 28.

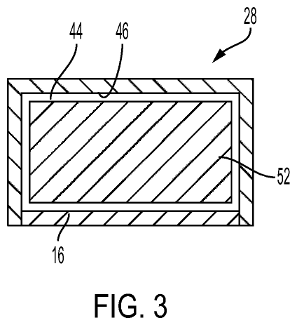

The first top plate 22 illustratively includes a C-channel 32 on the bottom surface 34 that extends a distance L1 (FIG. 1). The first top plate 22 illustratively includes an aperture 36 that aligns with the aperture 24 of the first end section 14 to allow the support 12 and the first top plate 22 to be assembled together via a fastener 38. The C-channel 32 is configured to receive a resorbable material insert 40 (FIGS. 1 and 3). As such, the resorbable material insert 40 pushes the first end section 14 downwardly away from the first top plate 22, wherein the fastener 38 through apertures 24 and 36 define a pivot point 42 of the first end section 14 relative to the first top plate 22.

The second top plate 28 illustratively includes a C-channel 44 on the bottom surface 46 that extends a distance L2 (FIG. 1). The second top plate 28 illustratively includes an aperture 48 that aligns with the aperture 30 of the second end section 16 to allow the support 12 and the second top plate 28 to be assembled together via a fastener 50. The C-channel 44 is configured to receive a resorbable material insert 52 (FIGS. 1 and 3). As such, the resorbable material insert 52 pushes the second end section 16 downwardly away from the second top plate 28, wherein the fastener 50 through apertures 30 and 48 define a pivot point 54 of the second end section 16 relative to the second top plate 28 (FIG. 2).

As noted above, the inserts 40 and 52 are formed of resorbable material. Resorbable materials have a wide range of material properties and resorption mechanics. Their relative tailorability makes them desirable for a wide range of situations and applications.

Resorbable material selection is an important factor when considering resorbable actuation. The type of resorbable material (PLA, tricalcium phosphate, etc.) as well as the medium in which it dissolves (saline solution, body fluid, water, etc.) greatly affects resorption mechanics. The material may reduce in volume while maintaining material properties (as is the case with rock salt) or may maintain the original volume but lose material properties like tensile strength (as is the case with most polymers). Furthermore, resorption times can range from several seconds to days to years depending on the selected material and solvent. Additional creativity can be achieved when different types of resorbable materials are layered or otherwise combined, allowing for truly unique resorption behavior.

Many resorbable materials dissolve at a rate proportional to the area exposed to the solvent. Thus, shapes with high surface area to volume ratios (SA:V) lead to quicker dissolution and quicker mechanism actuation.

The size of the resorbable material also affects resorption mechanics. The more resorbable material used, the longer it will take to dissolve. Thus if longer time before the mechanism actuation is desired, but a specific resorbable material must be used, the volume of the resorbable material can simply be increased to achieve the objective.

Like other materials, resorbable materials may be loaded in tension, compression, pure bending, torsion, direct shear, and combinations thereof. The loading type often determines the failure mode of the material (abrupt or gradual), which in turn governs the response of the mechanism.

The resorbable material of inserts 40 and 52 of the bar 10 are illustratively bioresorbable materials since they are configured to be positioned within the human body and dissolve as a result of exposure to bodily fluids (e.g., blood). Illustrative bioresorbable materials 40 and 52 are detailed in below Table 1.

TABLE 1
Various relevant bioresorbable materials
and their dissolution times in the body.

| Material | | | Time to Loss of | |
|---|---|---|---|---|
| Name | Dissolution Time | | Total Strength | |
| PGA | 17% degradation at 220 days or | | 1 | month |
| | 3-4 months | | | |
| PLA | 43% degradation at 220 days or 10 | | 3 | months |
| | months to 4 years or >24 months | | | |
| PLLA | 5.6 years or >24 months | | 3 | months |
| PCL | 6 months to 3 years or 92% excreted | | >6 | months |
| | by 135 days or >24 months | | | |
| PMMA | between 4 and 24 weeks | | between 1 and | |
| (CPC?) | | | 24 weeks | |
| Mg | 0.25 | months | <1 | month |
| TCP | >24 | months | 1-6 | months |

| | | | |
|---|---|---|---|
| Cellulose | highly dependent on material structure | <24 | months |
| | and environmental conditions | | |
| Silk | highly dependent on material structure | >7 | days |
| | and environmental conditions | | |

These bioresorbable materials may include polyglycolide (PGA), polylactic acid (PLA), poly-L-lactic acid (PLLA), polycaprolactone (PCL), polymethyl methacrylate (PMMA)(also commonly known as acylic), magnesium (Mg), tricalcium phosphate (TCP), cellulose and silk. For example, in medical device applications, polyglycolide (PGA), polylactic acid (PLA), tricalcium phosphate (TCP) and/or calcium carbonate may illustrative be used as bioresorbable materials. The listed dissolution times and times to loss of total strength in Table 1 are provided for illustrative purposes and have been supplied from various experimental sources. The values may vary widely based on the material structure, size, and environmental conditions.

With further reference to FIGS. 1 and 2, the lower support 12 illustratively has greater elasticity properties than that of the top plates 22 and 28. Further, the center section 18 is illustratively more flexible than the end sections 14 and 16. The relative elasticity properties (i.e., flexibility) of the support sections 14, 16, 18, and the top plates 22, 28 facilitate the device 10 achieving its final shape or undeformed position (i.e., activated mode) (FIGS. 2 and 10). In one illustrative embodiment, the lower support 12 may be formed of a biocompatible material, such as titanium-64 or 316 stainless steel. More particularly, the lower support 12 may be formed of titanium-64 as it is biocompatible and has enhanced compliant properties. The top plates 22 and 28 may be formed of a stiffer biocompatible material such as 316 stainless steel.

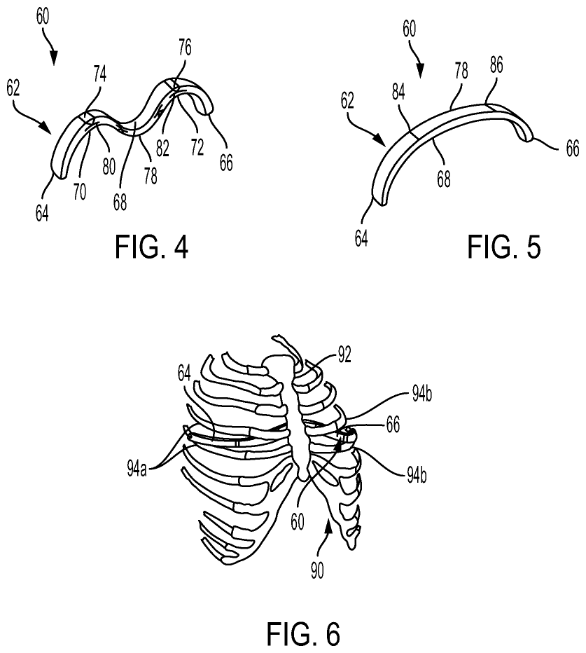

As shown in FIG. 4, a further illustrative embodiment shape-change mechanism of the present disclosure is shown in the form of a corrective device or bar 60 including a support 62 having a first end section 64, a second end section 66, and a middle or center section 68. Illustratively, the support 62 is formed of a flexible medical grade metal, such as titanium 64. In a first or stored energy mode, the support 62 is curved creating a downwardly facing first opening 70 between the first end section 64 and the center section 68, and a downwardly facing second opening 72 between the second end section 66 and the center section 68. The first end section 64 opposite the first opening 70 receives a first insert or wedge 74 made of a resorbable material. The second end section 66 opposite the second opening 72 receives a second insert or wedge 76 made of a resorbable material. The center section 68 is curved downwardly to define an upwardly facing center opening 78 intermediate the first opening 70 and second opening 72. Illustratively, microscopic slits 80 and 82 may be formed within the support 62 below the wedges 74 and 76, respectively, to improve flexibility of the support 62 and facilitate curving thereof.

FIG. 4 illustrates the corrective device 60 in a stored energy mode with resorbable inserts 74 and 76 in place to define the arcuate shape of the support 62 (i.e., deformed position). FIG. 5 illustrates the device 60 in an activated mode with the resorbable inserts 74 and 76 dissolved (i.e., natural, undeformed position). FIG. 6 is a perspective view of the illustrative device 60 in the mode of FIG. 4 and inserted through human rib cage 90 including a sternum 92 and ribs 94. More particularly, the center section 68 may engage the sternum 92, while the first and second end sections 64 and 66 may be supported by opposing ribs 94a and 94b. It should be appreciated that the device 10 may be positioned within the rib cage 90 in a similar manner.

As with the resorbable inserts 40 and 52 above, the inserts 74 and 76 are illustratively bioresorbable materials. As such, illustrative bioresorbable materials of the inserts 74 and 76 are detailed in above Table 1. For example, in medical device applications, polyglycolide (PGA), polylactic acid (PLA), tricalcium phosphate (TCP) and/or calcium carbonate could be used as bioresorbable materials.

With further reference to FIG. 4, the middle section 68 of the support 62 is illustratively more flexible than the end sections 64 and 66. The relative elasticity properties (i.e., flexibility) of the components 64, 66 and 68 facilitate the device 60 achieving its final shape (or activated mode) (FIG. 5). In one illustrative embodiment, the support 62 may be formed of a biocompatible material, such as titanium-64 or 316 stainless steel.

Table 2 below includes illustrative experimental observations.

TABLE 2
Experimental Observations

| Round | Description | Observation(s) |
|---|---|---|
| 1 | Proof of Concept | The bar 10, 60 deploys as expected |
| 2 | Mechanics of bioresorbable | Compressive strength during |
| | materials under different | resorption |
| | loading conditions | Compressive and tensile strength |
| | | after resorption |

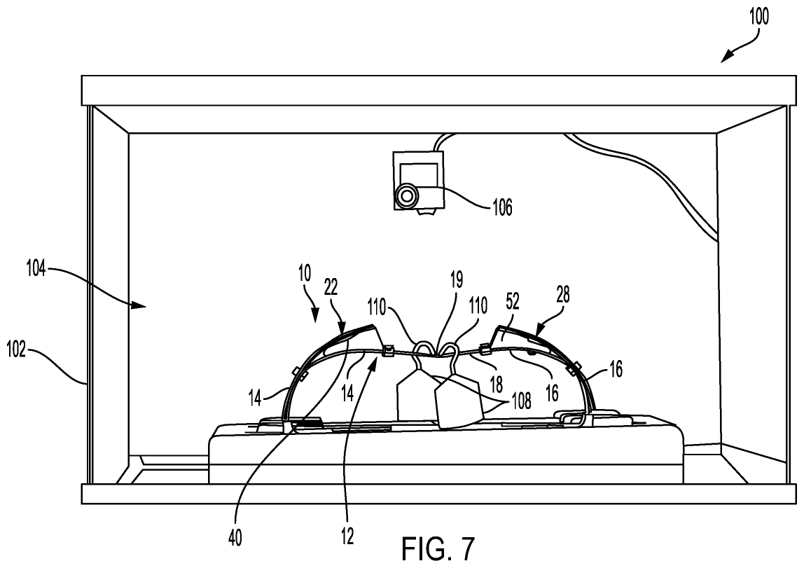

FIGS. 7-10 show a prototype of illustrative corrective device 10 of FIGS. 1-3 in a test environment or set-up 100. The test is set up to simulate in vivo conditions including the force that will be applied to the support 12 via a human sternum 92 when the device 10 is installed to repair pectus excavatum. It should be appreciated that the test set-up of FIGS. 7-10 is not an exact replica of in vivo conditions. The support 12 was made of aluminum (due to ease of manufacture) and the inserts 40 and 52 were made of rock salt (for rapid resorption, wherein actual materials would likely be designed to resorb over the course of months). The solvent used was warm water 104. The force shown was less than that predicted to be applied by a patient's sternum 92 for in vivo conditions, but was selected to be within the capabilities of the mock aluminum support 12.

The illustrative test set-up 100 includes a tank 102 holding water 104, illustratively 10 gallons at 98.6 F.° defining the bioresorbable solvent and simulate in vivo conditions. A pump 106 is configured to circulate the water 104 within the tank 102. Illustratively two 200 gram weights 108 simulate force of the sternum 92 and are coupled via hooks 110 proximate the center 19 of the support 12.

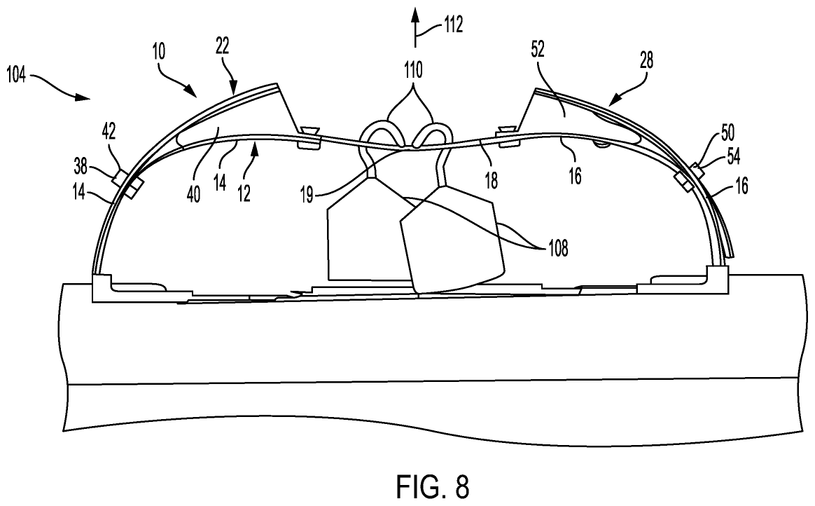

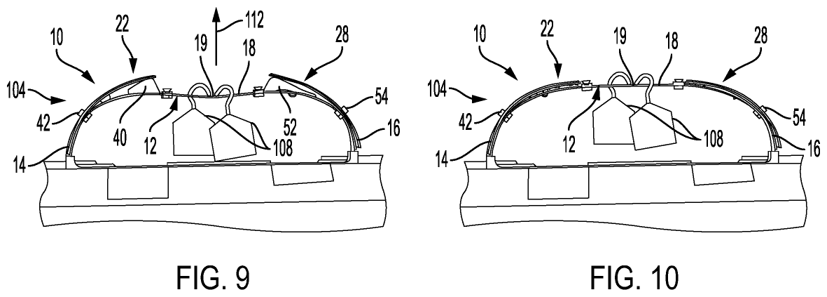

FIGS. 7 and 8 show inserts 40 and 52 made of bioresorbable material assembled into the support 12 intermediate the end sections 14, 16 and the top plates 22, 28, respectively. The support 12 is in a curved first position (stored energy mode), as described above. As shown in FIGS. 8 and 9, after the inserts 40 and 52 have partially dissolved, the support 10 becomes less curved in nature. The center point 19 of middle section 18 begins to rise as shown by arrow 112 from its first position (FIG. 7) to its second position (FIG. 10). With further reference to FIG. 10, once the inserts 40 and 52 have fully dissolved, the support 10 is in the second position (activated mode) as described above. The support 10 becomes arcuate to match the shape of a corrected sternum 92 and the center point 19 of middle section 18 becomes the apex of the support 10.

Table 3 shows illustrative model predictions.

TABLE 3
Illustrative Model Predictions

| Model | Predictions |
|---|---|
| Assume two pre-cured | Model Predicts |
| beams joined by a low | Maximum deformation before yielding |
| stiffness joint (LET | Force-deflection behavior |
| joint) | Model can include over-correction |
| Preliminary Model | 45%-50% initial correction |
| | As resorption occurs, 70%-80% correction |
| | detained assuming the chest does not |
| | reduce stiffness |
| | Assuming chest wall stiffness decreases, |
| | 95%-105% correction obtained |

Table 4 shows illustrative observations from the illustrative test set-up 100.

TABLE 4
Illustrative Observations

| Benefits | Drawbacks |
|---|---|
| Different materials provide different | Rate of deployment unalterable |
| rates of deployment; rate of deployment | post-operation |
| widely alterable pre-operation | Increased manufacturing from |
| Few parts | Nuss bar (but decreased from |
| Similar to Nuss bar | many other concepts) |
| Maximum output force similar to Nuss | |

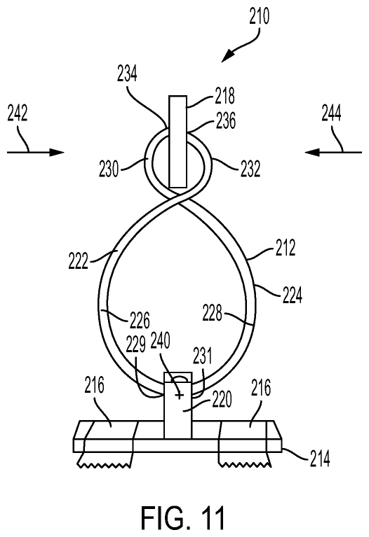

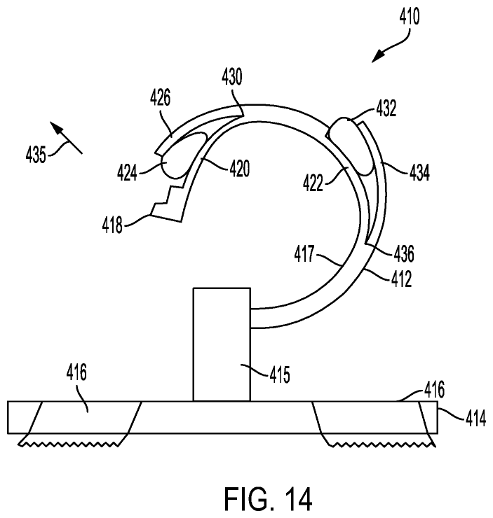

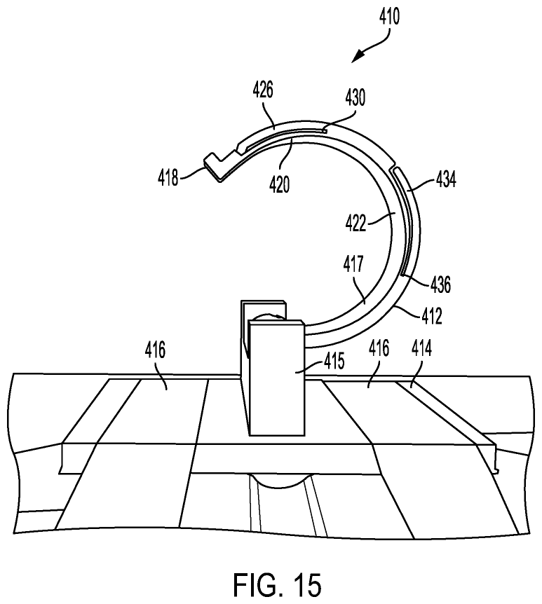

Two different actuation types are further detailed below: gradual and instantaneous (i.e. dynamic). For the instantaneous mechanism 210 of FIGS. 11-13, tension, torsion, bending, or direct shear would all be viable loading types. Direct shear was selected for the illustrative embodiment because it required the least amount of modification to the material and was the least affected by stress concentrations in the material. For the gradual mechanism 410 of FIGS. 14 and 15, the material was loaded in tension; this provided a straight-forward approach to actuating the mechanism as the volume of the resorbable material decreased. It should be noted that the shape-change mechanisms 10 and 60 as detailed above may also be characterized as gradual mechanisms.

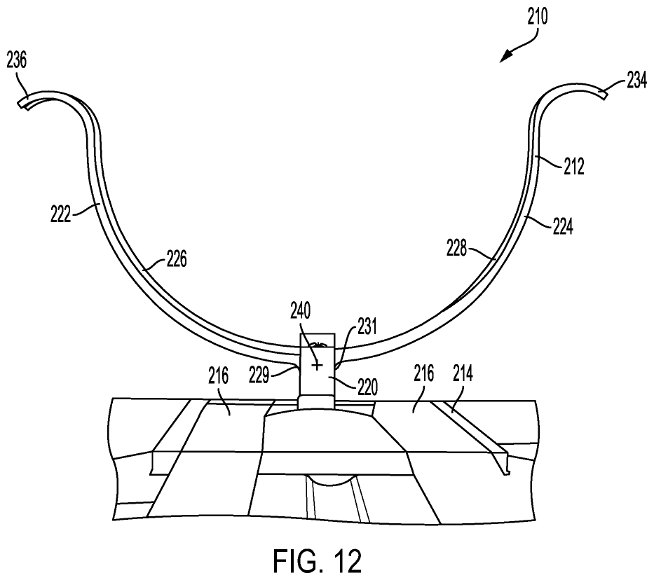

With further reference now to FIGS. 11 and 12, the illustrative shape change mechanism 210 is shown in the form of a compliant clamp 212. The compliant clamp 212 illustratively includes a resorbable material insert 218 cooperating with opposing first and second arms 222 and 224. A base 214 is illustratively secured to a support by couplers 216, such as adhesive tape. An upright 220 supports first or lower ends 229 and 231 of the arms 222 and 224 above the base 214. First and second curved portions 226 and 232 extend between the opposing ends 229 and 236 of the first arm 222. Similarly, first and second curved portions 228 and 230 extend between the opposing ends 231 and 234 of the second arm 224. The respective first curved portions 226, 228 and second curved portions 230, 232 face each other such that the first arm 222 is in the shape of a reverse “S” and the second arm 224 is in the shape of an “S” in the deformed position of FIG. 11. In other words, the first curved portion 226 faces the opposite direction from the first curved portion 228 and the second curved portion 232, and the second curved portion 230 faces the opposite direction from the first curved portion 228 and the second curved portion 232.

The resorbable material insert 218 is positioned intermediate the opposing ends 234 and 236 of the arms 222 and 224. With further reference to FIG. 11, the compliant clamp 212 is shown in its deformed position where it is clamping the resorbable material insert 218 having a rectangular shape. The compliant arms 222 and 224 are bent to form the clamp 212, wherein the insert 218 of resorbable material is placed between the opposing ends 234 and 236, which locks the mechanism 210 in its deformed state. As the material of the insert 218 dissolves, its reduction in size weakens it until it catastrophically fails under the restoring forces of the clamp arms 222 and 224. With the obstruction 218 removed, the mechanism 210 returns immediately and completely to its relaxed, equilibrium state of FIG. 12. As such, this demonstrates that the slowly dissolving material of the insert 218 can be loaded in direct shear to serve as actuation for a dynamic, binary-state (clamped or open) device 210.

FIG. 11 illustrates the mechanism in a first or deformed position, while FIG. 12 illustrates the mechanism 212 in a natural, undeformed position. As the resorbable material 218 dissolves, the ends 234 and 236 move in opposing directions about pivot point 240 (as shown by arrows 242 and 244). As shown, the ends 234 and 236 move from a first distance apart from each other in FIG. 11, to a second distance apart from each other in FIG. 12. The second distance is greater than the first distance. Additionally, the first end 234 moves from a left side of the pivot point 240 in FIG. 11 to a right side of the pivot point 240 in FIG. 12, while the second end 236 moves from a right side of the pivot point 240 in FIG. 11 to a left side of the pivot point 240 in FIG. 12.

During experiments, the device 210 was manufactured using a conventional additive material three-dimensional (3D) printer. It was tested four times, with almost identical behavior for each test. In all cases, the arms 222 and 224 move slightly as resorbable material of the insert 218 dissolved due to the smaller volume of the obstruction. Upon fracture of the resorbable material of the insert 218, the arms 222 and 224 of the clamp 212 dramatically flared out to the open position. This produced a delayed but sudden actuation of the clamping device 210.

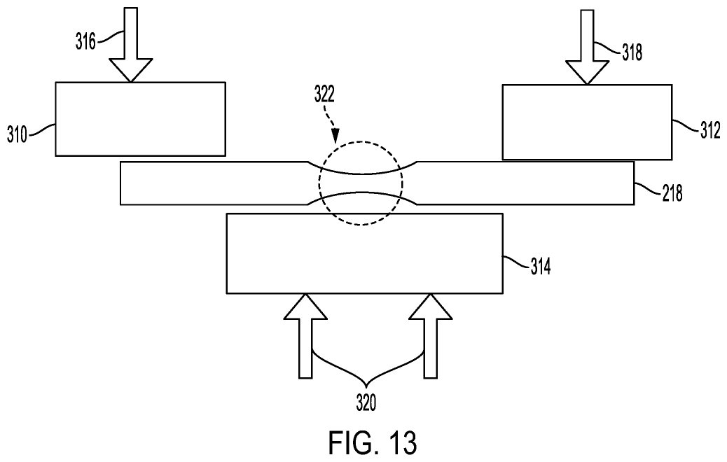

FIG. 13 illustrates a dog-bone shaped bioresornable material insert 218 of the type that may be used within the compliant clamp 212. A thinned out section 322 provides a failure point due to smaller cross-section. Opposing forces 316, 318 and 320 are illustratively applied to clamp members 310, 312 and 314, respectively.

The shape-change mechanism 210 demonstrated the ability to control the location of the breaking point of resorbable material by bearing its cross-sectional geometry. Resorbable inserts 218 having uniform cross-section, as well as inserts 218 that were thinned out in the center (FIG. 13) to see if it affected where the material failed. For the two thinned out samples, the inserts 218 broke in the middle 322 exactly where the material had been thinned out. This is in contrast to the insert 218 that had a uniform cross-section, where the fractured line was off-center (likely due to a crack earlier imperfection in the material before the testing commenced). The support of the concept of adding thin-out geometry for more consistent breakage of material, which mitigated the effects that imperfections and stock material had on resorption behavior.

The instantaneous shape-change mechanism 210: (1) shows that resorbable materials may be loaded in direct shear to actuate a mechanism; (2) shows that resorbable materials may be used to actuate a mechanism at a desired time (degradation time depends on material type, size, and surface area exposed to solvent, and so these qualities can be controlled to retrieve particular results); (3) shows that fracture location and resorbable materials can also be controlled-smallest cross-sectional area varied directly with the amount of time required for the solution (therefore, by creating thinner portions of the cross-section of the material (e.g., dog-bone shape), fracture location to specify for predictable failure); and (4) shows that resorbable material may dissolve at different rates based on their loading conditions.

Due to its nature as an auto-activation mechanism, the shape-change mechanism 210 can be used in a wide variety of situations that are largely removed from human interaction and control. These applications may include biomedical applications (e.g., in vivo actuation (e.g., stents)), self-repair applications (e.g., clamp could stop a leak as it deploys open), and deep sea applications (e.g., releasing a research submarine at specific time during the dive, and/or clamp holds a weight which weighs down the submarine for a specific amount of time until the material dissolves and the submarine is released). Additional potential applications may include space applications (locking onto a tube for a specified amount of time, automatic actuation of a mechanism (e.g., of a deployable solar array), and/or upon leaking of air from the spacecraft, the clamp could open up to stop or slow the leak), and electronics applications (a circuit that closes or opens once at a time determined by the device's internal temperature).

FIGS. 14 and 15 show another illustrative shape change mechanism, illustratively a gradual shape change mechanism 410 including an arcuate arm 412 coupled to a base 414 via an upright support 415. Couplers, such as adhesive tape 416, may secure the base 414 to a support (not shown). The arm 412 includes an inwardly facing arcuate surface 417.

The illustrative arm 412 includes a hook-like feature as an end effector 418. The arm 412 is designed to have first and second compliant sections 420 and 422 having reduced thicknesses, which allow for increased compliance in these areas, so that the end effector 418 has a longer travel distance from start to finish.

A first resorbable material insert 424 is coupled to the arm 412 in a pocket intermediate the inner surface 417 and an outer member 426 at the first compliant section 420. The outer member 426 is pivotably coupled to the arm 412 at a joint or hinge 430. A second resorbable material insert 432 is coupled to the arm 412 in a pocket intermediate the inner surface 417 and an outer member 434. The outer member 434 is pivotably coupled to the arm 412 at a joint or hinge 436.

Once the resorbable material inserts 424 and 432 are inserted into their respective spots in the mechanism 410, the end effector 418 is in a new position, and as resorbable material inserts 424 and 432 dissolve, the end effector 418 rotates and translates to a predictable path back to its original undeformed position (as indicated by arrow 435 in FIG. 14). More particularly, when the resorbable members 424 and 432 dissolve, the arm 412 moves from the position of FIG. 14 to the position of FIG. 15.

The mechanism 410 could alternatively be designed to produce a wide variety of motions that fit many different applications by varying the location and the thickness of the compliant sections 420 and 422, undeformed shape, material, and/or by adding more compliant sections 420 and 422.

A gradual shape-change mechanism: (1) shows a resorbable material that may be used in compression to actuate a mechanism; (2) shows that resorption materials may be used to produce gradual actuation, with a specified time interval between beginning and end positions; (3) shows that continuous actuations can be achieved using resorbable materials; and (4) shows that end effector paths may be pre-determined and specified by modifying the shape, size, and type of resorbable material.

Similar to the instantaneous shape change mechanism 210, the gradual shape change mechanism 410 may also be used in a variety of situations where human interaction (e.g., manual activation) is difficult or impossible. Gradual deployment also has many advantages over immediate deployment in several applications. These applications may include biomedical applications (e.g., gradual repair for deformities such as pectus excavatum, palette expansion, or scoliosis correction), and/or robotics applications (e.g., moving the end effector to desired position after a certain amount of time). The shape change mechanism 310 may also be used in space applications, including transitioning a switch between states at a specific time, moving masses outside of the spacecraft, and/or using many of these mechanisms in series to deploy an antenna from its stowed position.

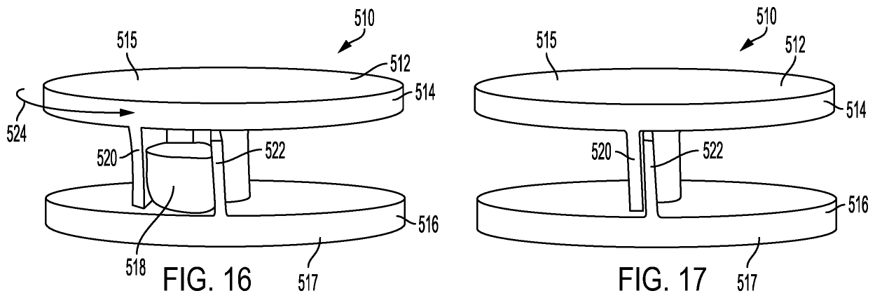

FIGS. 16 and 17 illustrate a shape change mechanism 510 that is configured to translate resorbsion of bioabsorable material to rotation. Illustratively, the actuation could function as a valve, or could align two holes (similar to twisting lids like those on scent products) to allow movement of a member through a blocking point.

With further reference to FIGS. 16 and 17, the rotary shape-change mechanism 510 illustratively includes a body 512 including an upper member or body 514 coupled to a lower member or body 516. The upper member 514 illustratively includes an upper plate 515 supporting a downwardly extending arm 520. The lower member 516 illustratively includes a lower plate 517 supporting an upwardly extending arm 522. A resorbable material insert 518 is positioned intermediate the arms 520 and 522. The upper member 514 and the lower member 516 are compliantly coupled such that the arms 520 and 522 are rotationally biased towards each other. This biasing may illustratively be accomplished through inherent material properties or a spring mechanism. As the resorbable material insert 518 dissolves, the arms 520 and 522 and therefore members 514 and 516 tend to move towards each other as shown by arrow 524 in FIG. 16. The deformed position is shown in FIG. 16, while the natural, undeformed position is shown in FIG. 17.

The above shape-change mechanisms 10, 60, 210, 410, 510 represent illustrative mechanical systems that are actuated using resorbable materials. Resorbable materials have significant shape-changing potential when combined with compliance mechanisms 10, 60, 210, 410, 510 like those detailed above. The resorbable material controls the release of the strain energy that acts to restore a bent compliant system to its original shape, which can result in either shapes that snap into place or slowly transforming geometries. Resorbable materials can actuate traditional mechanisms as well, such as the rotating mechanism detailed above.

It has been demonstrated that in tests that actuation of the mechanical systems using resorbable materials is possible and advantageous. It should be appreciated that the principles detailed herein may be utilized in a wide variety of shape change mechanisms.

Although the invention has been described in detail with reference to certain preferred embodiments, variations and modifications exist within the spirit and scope of the invention as described and defined in the following claims.

What is claimed is:

1. A resorbable material activated device comprising:
a support including a first end section, an opposing second end section, and a center section intermediate the first end section and the second end section;
a first resorbable material positioned intermediate the first end section and the center section of the support;
a second resorbable material positioned intermediate the second end section and the center section of the support; and
wherein the shape of the support changes in response to dissolving of the first and second resorbable materials.

2. The device of claim 1, wherein the center section moves from a first position to a second position in response to dissolving of the first and second resorbable materials.

3. The device of claim 1, wherein the support comprises a biocompatible material.

4. The device of claim 3, wherein the biocompatible material is at least one of titanium or stainless steel.

5. The device of claim 1, wherein the center section has greater elasticity than the first and second end sections.

6. The device of claim 1, wherein the support is insertable within the rib cage of a human for repairing pectus excavatum.

7. The device of claim 1, wherein:
the first end section includes a first cutout;
a first top plate is supported in the first cutout and includes a first channel receiving the first resorbable material;
the second end section includes a second cutout; and
a second top plate is supported in the second cutout and includes a second channel receiving the second resorbable material.

8. The device of claim 7, further comprising:
a first pivot point coupling the first top plate to the first end section; and
a second pivot point coupling the second top plate to the second end section.

9. The device of claim 1, wherein each of the first and second resorbable materials includes at least one of polyglycolide (PGA), polylactic acid (PLA), tricalcium phosphate (TCP) or calcium carbonate.

10. A resorbable material activated device comprising:
a compliant segment including opposing first and second ends;
a resorbable material operably coupled to the compliant segment intermediate the first and second ends; and
wherein at least one of the shape, position or configuration of the compliant segment changes in response to dissolving of the resorbable material.

11. The device of claim 10, wherein the center section moves from a first position to a second position in response to dissolving of the first and second bioresorbable materials.

12. The device of claim 10, wherein the support comprises a biocompatible material.

13. The device of claim 12, wherein the biocompatible material is at least one of titanium or stainless steel.

14. The device of claim 10, wherein the center section has greater elasticity than the first and second end sections.

15. The device of claim 10, wherein the support is insertable within the rib cage of a human for repairing pectus excavatum.

16. The device of claim 10, wherein:
the first end section includes a first cutout;
a first top plate is supported in the first cutout and includes a first channel receiving the first bioresorbable material;
the second end section includes a second cutout; and
a second top plate is supported in the second cutout and includes a second channel receiving the second bioresorbable material.

17. The device of claim 16, further comprising:
a first pivot point coupling the first top plate to the first end section; and
a second pivot point coupling the second top plate to the second end section.

18. The device of claim 10, wherein each of the first and second bioresorbable materials includes at least one of polyglycolide (PGA), polylactic acid (PLA), tricalcium phosphate (TCP) or calcium carbonate.

19. A device for repairing pectus excavatum in a human body, the device comprising:
a support including a first end section, an opposing second end section, and a center section intermediate the first end section and the second end section, the support formed of a biocompatible material;
a first bioresorbable material positioned intermediate the first end section and the center section of the support;
a second bioresorbable material positioned intermediate the second end section and the center section of the support; and
wherein the center section moves from a first position to a second position in response to dissolving of the first and second bioresorbable materials to exert a force against a sternum of the human body.

20. The device of claim 19, wherein the biocompatible material is at least one of titanium or stainless steel.

21. The device of claim 19, wherein the center section has greater elasticity than the first and second end sections.

22. The device of claim 19, wherein the support is insertable within the rib cage of a human for repairing pectus excavatum.

23. The device of claim 19, wherein:
the first end section includes a first cutout;
a first top plate is supported in the first cutout and includes a first channel receiving the first bioresorbable material;
the second end section includes a second cutout; and
a second top plate is supported in the second cutout and includes a second channel receiving the second bioresorbable material.

24. The device of claim 23, further comprising:
a first pivot point coupling the first top plate to the first end section; and
a second pivot point coupling the second top plate to the second end section.

25. The device of claim 19, wherein each of the first and second bioresorbable materials includes at least one of polyglycolide (PGA), polylactic acid (PLA), tricalcium phosphate (TCP) or calcium carbonate.

26. A method of repairing pectus excavatum in a human body, the method comprising the steps of:
providing a support including a first end section, an opposing second end section, and a center section intermediate the first end section and the second end section;
providing a first bioresorbable material intermediate the first end section and the center section of the support;
providing a second bioresorbable material intermediate the second end section and the center section of the support;
inserting the support within a rib cage of the human body; and
dissolving the first and second bioresorbable materials resulting in the changing the shape of the support in response to dissolving of the first and second bioresorbable materials.

27. The method of claim 26, wherein the shape changing step includes moving the center section from a first position to a second position to exert a force against a sternum of the rib cage.

28. The method of claim 26, wherein the biocompatible material is at least one of titanium or stainless steel.

29. The method of claim 26, wherein the center section has greater elasticity than the first and second end sections.

30. The method of claim 26, wherein:
the first end section includes a first cutout;
a first top plate is supported in the first cutout and includes a first channel receiving the first bioresorbable material;
the second end section includes a second cutout; and
a second top plate is supported in the second cutout and includes a second channel receiving the second bioresorbable material.

31. The method of claim 30, further comprising:
a first pivot point coupling the first top plate to the first end section; and
a second pivot point coupling the second top plate to the second end section.

32. The method of claim 26, wherein each of the first and second bioresorbable materials includes at least one of polyglycolide (PGA), polylactic acid (PLA), tricalcium phosphate (TCP) or calcium carbonate.

33. A resorbable material activated device comprising:
a first compliant arm extending between a first end and a second end, the first compliant arm including a first curved portion and a second curved portion intermediate the first end and the second end, the first curved portion facing an opposite direction from the second curved portion;
a second compliant arm extending between a first end and a second end, the second compliant arm including a first curved portion and a second curved portion intermediate the first end and the second end, the first curved portion facing an opposite direction from the second curved portion;
wherein the second ends of the first compliant arm and the second compliant arm are positioned a first distance apart from each other in a deformed mode;
wherein the second ends of the first compliant arm and the second compliant arm are positioned a second distance apart from each other in a natural, undeformed mode, the second distance being greater than the first distance;
a resorbable material insert positioned intermediate the second ends of the first compliant arm and the second compliant arm in the deformed mode; and
wherein the resorbable material insert is removed from intermediate the second ends of the first compliant arm and the second compliant arm in the natural, undeformed mode.

34. The resorbable material activated device of claim 33, wherein the second ends of the first compliant arm and the second compliant arm move about a pivot point between the deformed mode and the natural, undeformed mode.

35. The resorbable material activated device of claim 33, further comprising a solvent to dissolve the resorbable material insert.

36. The resorbable material activated device of claim 33, wherein the resorbable material insert includes a reduced cross-sectional area.

37. A resorbable material activated device comprising:
an arcuate arm including a first compliant section defining a first pocket;
an end effector supported by the arcuate arm;
a first resorbable material insert supported within the first pocket in a deformed mode, and removed from the first pocket in a natural, undeformed mode;
wherein the end effector is in a first position in the deformed mode; and
wherein the end effector is in a second position in the natural, undeformed mode.

38. The resorbable material activated device of claim 37, wherein:
the arcuate arm includes a second compliant section defining a second pocket; and
a second resorbable material insert is supported within the second pocket in the deformed mode, and is removed from the second pocket in the natural, undeformed mode.

39. The resorbable material activated device of claim 37, further comprising a solvent to dissolve the first resorbable material insert.
